(19) United States
(12) Patent Application Publication
(10) Pub. No.: US 2011/0268794 A1
(43) Pub. Date: Nov. 3, 2011

(54) METHODS AND MATERIALS FOR DELIVERING BILE ACIDS

(76) Inventors: Michael L. Camilleri, Rochester, MN (US); Duane D. Burton, Kasson, MN (US)

(21) Appl. No.: 13/143,640

(22) PCT Filed: Jan. 4, 2010
(86) PCT No.: PCT/US10/20042

§ 371 Date: Jul. 7, 2011

(30) Foreign Application Priority Data
Jan. 9, 2009 (US) 61143727

(51) Int. Cl. A61K 31/575; A61K 9/48; A61P 1/00

(52) U.S. Cl. 424/456; 514/182

(57) ABSTRACT

This document relates to methods and materials for administering bile acid compounds to treat conditions associated with constipation. For example, formulations designed for the delayed-release of a bile acid compound (e.g., sodium chenodeoxycholate) to treat constipation are provided.

### CROSS-REFERENCE TO RELATED APPLICATIONS

This application claims priority to U.S. Provisional Application Ser. No. 61/143,727, filed on Jan. 9, 2009.

### BACKGROUND

1. Technical Field

This document relates to methods and materials for administering bile acid compounds to treat conditions associated with constipation. For example, this document provides formulations designed for the delayed-release of a bile acid compound (e.g., sodium chenodeoxycholate) to treat a condition associated with constipation (e.g., occasional constipation, chronic constipation, functional constipation, opiate-induced constipation, chronic colonic pseudoobstruction, slow transit constipation, or colonic inertia).

2. Background Information

Bile acids are formed in the liver from cholesterol and have a variety of physiologic functions from cholesterol elimination to enhancement of lipid absorption in the small intestine. Up to 95% of bile acids secreted into bile are actively reabsorbed in the terminal ileum.

### SUMMARY

This document relates to methods and materials for administering bile acid compounds to treat conditions associated with constipation. For example, this document provides formulations designed for the delayed-release of a bile acid compound (e.g., sodium chenodeoxycholate) to treat a condition associated with constipation (e.g., occasional constipation, chronic constipation, functional constipation, opiate-induced constipation, chronic colonic pseudoobstruction, slow transit constipation, or colonic inertia).

In general, one aspect of this document features a method for treating a constipation condition. The method comprises, or consists essentially of, administering to a mammal having a constipation condition a composition comprising a bile acid compound, wherein the composition is configured for the delayed-release of the bile acid compound to the ileocolonic region of the mammal. The mammal can be a human. The constipation condition can be occasional constipation, chronic constipation, functional constipation, opiate-induced constipation, chronic colonic pseudoobstruction, slow transit constipation, or colonic inertia. The bile acid compound can be sodium chenodeoxycholate. The composition can comprise, or consist essentially of, sodium chenodeoxycholate coated with methacrylate.

In another aspect, this document features a composition comprising a bile acid compound (e.g., a sodium chenodeoxycholate) coated with a pH sensitive polymer (e.g., methacrylate) such that the bile acid compound (e.g., sodium chenodeoxycholate) is capable of being delivered to the ileocolonic region of a mammal following an oral administration. The mammal can be a human. The composition can comprise between 250 and 5000 mg of sodium chenodeoxycholate. The composition can comprise between 500 and 1500 mg of sodium chenodeoxycholate. The composition can comprise about 1000 mg of sodium chenodeoxycholate. The coating can be between 10 μm and 90 μm in thickness. The coating can be between 12 μm and 75 μm in thickness. The coating can be between 40 μm and 60 μm in thickness. The coating can be about 50 μm in thickness. The bile acid compound (e.g., sodium chenodeoxycholate) can be within a gelatin capsule, and the gelatin capsule can be coated with the methacrylate. The gelatin capsule can have a thickness of between about 100 μm and about 160 μm. The gelatin capsule can have a thickness of between about 110 μm and about 150 μm. The gelatin capsule can have a thickness of between about 120 μm and about 140 μm.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1 is a diagram of the study design for the study of Example 1.

FIG. 2 is a flow chart of the study of Example 1.

FIG. 3 is a graph plotting the effect of chenodeoxycholate on colonic transit (GC) at 24 and 48 hours, and stool form and frequency. Data show least square means±SEM.

### DETAILED DESCRIPTION

This document relates to methods and materials for administering bile acid compounds to treat conditions associated with constipation. For example, this document provides compositions designed for the delayed-release of one or more bile acid compounds (e.g., sodium chenodeoxycholate) to treat a condition associated with constipation (e.g., occasional constipation, chronic constipation, functional constipation, opiate-induced constipation, chronic colonic pseudoobstruction, slow transit constipation, or colonic inertia). Such compositions can be formulated for the delayed-release to the ileocolonic region of a mammal (e.g., a human, dog, cat, horse, pig, monkey, or sheep) using, for example, one or more pH sensitive polymers (e.g., methacrylate). Examples of bile acid compounds include, without limitation, sodium or potassium chenodeoxycholate, sodium or potassium glycochenodeoxycholate, sodium or potassium taurochenodeoxycholate, sodium or potassium deoxycholate, sodium or potassium glycodeoxycholate, sodium or potassium taurodeoxycholate, sodium or potassium cholate, sodium or potassium glycocholate, and sodium or potassium taurocholate.

Any appropriate method can be used to formulate one or more bile acid compounds into a composition designed to deliver the bile acid compounds to the ileocolonic region of a mammal. For example, a composition provided herein can be designed to contain one or more bile acid compounds and to have a coating that prevents release of the one or more bile acid compounds until the formulation reaches the ileocolonic region of a mammal following oral administration. In general, a coating can be provided on a capsule, tablet, or pellet containing one or more bile acid compounds to prevent release until the tablet, capsule, or pellet reaches the ileocolonic region of a mammal. Any appropriate coating can be used to allow bile acid compounds to be delivered to the ileocolonic region including, without limitation, pH sensitive coatings, redox sensitive coatings, and coatings sensitive to enzymes or bacteria.

In some cases, a pH sensitive coating that can be used to make a composition provided herein can include a material that dissolves at a pH of 5 or above (e.g., pH of 5 or more, 5.5 or more, 6.0 or more, 6.5 or more, or 7.0 or more). Such a coating can begin to dissolve when it exits the stomach and enters the small intestine. A thick layer of the coating can be used such that the coating dissolves in about three to four hours, thereby allowing the bile acid compounds underneath (e.g., a capsule of bile acid compounds underneath) to breakup when it reaches the ileocolonic region. In general, as the pH at which the coating begins to dissolve increases, the thickness necessary to achieve delivery to the ileocolonic region decreases. Examples of materials that can be used to make a pH sensitive coating of a composition provided herein include, without limitation, methacrylate, methylmethacrylates, copolymers of methacrylic acid and methylmethacrylate, cellulose acetate trimellitate (CAT), hydroxypropylmethyl cellulose phthalate (HPMCP), polyvinyl acetate phthalate (PVAP), cellulose acetate phthalate (CAP), and shellac.

As described herein, a redox-sensitive coating can be used to make a composition such that a bile acid compound is delivered to the ileocolonic region. Examples of materials that can be used to make a redox-sensitive coating include, without limitation, azopolymers and disulphide polymers. Azopolymers can consist of a random copolymer of styrene and hydroxyethyl methacrylate, cross-linked with divinylazobenzene synthesized by free radical polymerization. In some cases, a composition containing one or more bile acid compounds can be formulated for delivery to the ileocolonic region of a mammal's digestive tract using the methods and materials described elsewhere (see, e.g., U.S. Pat. No. 5,407,682 and Van den Mooter, Int. J. Pharm., 87:37 (1992)).

In some cases, amylose, cellulose, acrylic polymer materials, calcium pectinate, pectin, chondroitin sulphate, resistant starches, dextran hydrogels, modified guar gum (e.g., borax modified guar gum), β-cyclodextrin, time release systems, or combinations thereof can be used to formulate a composition such that a bile acid compound is delivered to the ileocolonic region as described elsewhere (see, e.g., U.S. Pat. Nos. 4,871,549; 5,294,448; 6,200,602; 6,350,471; and 7,612,112).

Any appropriate method can be used to obtain coating materials and to formulate a composition for delivery to the ileocolonic region using coating materials. For example, a composition containing a bile acid compound can be formulated for delivery to the ileocolonic region of a mammal's digestive tract by coating a capsule containing 1000 mg of sodium chenodeoxycholate with methacrylate. Any appropriate thickness can be used to allow for delivery to the ileocolonic region including, for example, a thickness between about 5 μm and about 100 μm (e.g., between about 10 μm and about 100 μm, between about 15 μm and about 100 μm, between about 20 μm and about 100 μm, between about 25 μm and about 100 μm, between about 10 μm and about 90 μm, between about 10 μm and about 80 μm, between about 10 μm and about 70 μm, between about 10 μm and about 60 μm, between about 12 μm and about 75 μm, between about 15 μm and about 75 μm, between about 20 μm and about 70 μm, between about 30 μm and about 70 μm, between about 40 μm and about 60 μm, or between about 45 μm and about 55 μm). In some cases, the average thickness of a coating (e.g., a methacrylate coating) can be about 30, 35, 40, 45, 50, 55, 60, 65, 70, or 75 μm. In some cases, a capsule containing one or more bile acid compounds (e.g., sodium chenodeoxycholate) can be made to have a coating (e.g., a methacrylate coating) using a dipping process. For example, a capsule containing sodium chenodeoxycholate can be coated via a single 5 second dip into a solution having methacrylate (e.g., 13 g of methacrylate in 100 mL of a 4:6 ratio of acetone to isopropyl alcohol) as described elsewhere (Proano et al., Am. J. Physiol., 258 (Gastrointest. Liver Physiol., 21): G856-G862 (1990)). The capsule can be a gelatin capsule having a thickness between about 60 μm and about 200 μm (e.g., between about 60 μm and about 200 μm, between about 60 μm and about 180 μm, between about 60 μm and about 160 μm, between about 60 μm and about 150 μm, between about 70 μm and about 200 μm, between about 90 μm and about 200 μm, between about 110 μm and about 200 μm, between about 120 μm and about 200 μm, between about 100 μm and about 160 μm, between about 110 μm and about 150 μm, between about 120 μm and about 140 μm). In some cases, the capsule can be a gelatin capsule having a thickness of about 110, 120, 130, 140, or 150 μm. In some cases, a composition containing one or more bile acid compounds can be formulated for delivery to the ileocolonic region of a mammal's digestive tract using the methods and materials described elsewhere (see, e.g., Healy “Enteric Coatings and Delayed Release” Chapter 7 in Drug Delivery to the Gastrointestinal Tract, editors Hardy et al., Ellis Horwood, Chichester, 1989 and U.S. Pat. No. 6,200,602).

A composition containing a bile acid compound can be administered to a mammal in any amount, at any frequency, and for any duration effective to achieve a desired outcome (e.g., to treat constipation). In some cases, a composition containing a bile acid compound can be administered to a mammal to increase colonic transit by 1, 5, 10, 11, 12, 13, 14, 15, 16, 17, 18, 19, 20, 25, 30, 35, 40, 45, 50, 55, 60, 65, 70 percent or more). An effective amount of a composition containing a bile acid compound can be any amount that reduces a mammal's constipation without producing significant toxicity to a mammal. Typically, an effective amount of a composition containing a bile acid compound can be any amount greater than or equal to about 250 mg of a bile acid compound (e.g., greater than or equal to about 250, 500, 750, 1000, 1250, 1500, 1750, 2000, or more mg of, for example, sodium chenodeoxycholate per administration) provided that that amount does not induce significant toxicity to the mammal upon administration. In some cases, an effective amount of a bile acid compound such as sodium chenodeoxycholate can be between 250 mg and 5000 mg (e.g., between 250 mg and 1250 mg, between 500 mg and 1500 mg, or between 750 mg and 2000 mg). Various factors can influence the actual effective amount used for a particular application. For example, the frequency of administration, duration of treatment, use of multiple treatment agents, route of administration, and severity of the constipation may require an increase or decrease in the actual effective amount administered.

The frequency of administration of a composition containing a bile acid compound can be any frequency that reduces a mammal's constipation without producing significant toxicity to the mammal. For example, the frequency of administration can be from about three times a day to about twice a week (e.g., once a day). The frequency of administration can remain constant or can be variable during the duration of treatment. For example, a composition containing a bile acid compound can be administered daily, twice a day, five days a week, or three days a week. A composition containing a bile acid compound can be administered for five days, 10 days, three weeks, four weeks, eight weeks, 48 weeks, one year, 18 months, two years, three years, or five years. A course of treatment can include rest periods. For example, a composition containing a bile acid compound can be administered for five days followed by a ten-day rest period, and such a regimen can be repeated multiple times. As with the effective amount, various factors can influence the actual frequency of administration used for a particular application. For example, the effective amount, duration of treatment, use of multiple treatment agents, route of administration, and severity of the constipation may require an increase or decrease in administration frequency.

An effective duration for administering a composition containing a bile acid compound can be any duration that reduces a mammal's constipation without producing significant toxicity to the mammal. Thus, the effective duration can vary from several days to several weeks, months, or years. In general, the effective duration for the treatment of constipation can range in duration from one day to several days to several months. In some cases, an effective duration can be for as long as an individual mammal is alive and suffering from constipation. Multiple factors can influence the actual effective duration used for a particular treatment. For example, an effective duration can vary with the frequency of administration, effective amount, use of multiple treatment agents, route of administration, and severity of the constipation.

Once orally administered to a mammal (e.g., a human), the mammal can be assessed to confirm a reduction in the mammal's constipation condition. For example, colonic transit and/or stool consistency can be assessed using standard techniques such as those described herein to confirm a reduction in the mammal's constipation condition. In some cases, human patients orally administering a composition provided herein can be asked to confirm an improvement with their constipation conditions.

The invention will be further described in the following examples, which do not limit the scope of the invention described in the claims.

### EXAMPLES

### Example 1

### Effects of Delayed-Release Chenodeoxycholate on Gastrointestinal and Colonic Transit and Bowel Function in Healthy Volunteers

### Study Design, Randomization, and Medication

This was a double-blind, placebo-controlled, randomized study evaluating the effects of sodium chenodeoxycholate (CDC) or placebo orally administered once daily for four days in healthy volunteers.

Randomization was 1:1:1 for placebo, chenodeoxycholate 500 mg, and chenodeoxycholate 1000 mg. Sodium chenodeoxycholate was purchased from Calbiochem, EMD Chemicals Inc., and Mayo pharmacy prepared identical placebo and chenodeoxycholate capsules, all of which were coated with the pH sensitive polymer, methacrylate. The latter dissolves at the neutral pH found in the distal ileum and was used to ensure ileocolonic delivery of the chenodeoxycholate.

An independent statistician generated the randomization code. Clinical and laboratory study personnel were blinded throughout the study until data were locked and analyzed. Safety monitoring was conducted throughout the study.

### Participants

60 patients (mean age 38.7 years, 43 female) were enrolled to the study. Participants had fasting plasma 7α-HCO(C4) measured to assess for underlying bile acid malabsorption, Hospital anxiety and depression (HAD) scale, SCL-90 (somatization) (Zigmond and Snaith, Acta Psychiatr. Scand., 67:361-70 (1983)), and bowel function (by validated daily diaries including Bristol Stool Form Scale (BSFS) scores (Bouras et al., Gastroenterology, 120:354-60 (2001); and Lewis and Heaton, Scand. J. Gastroenterol., 32:920-4 (1997)).

Three groups (n=20 each) were randomized to oral placebo, 500 mg or 1000 mg CDC daily (in pH-sensitive, methacrylate-coated capsules), each for a period of 4 days.

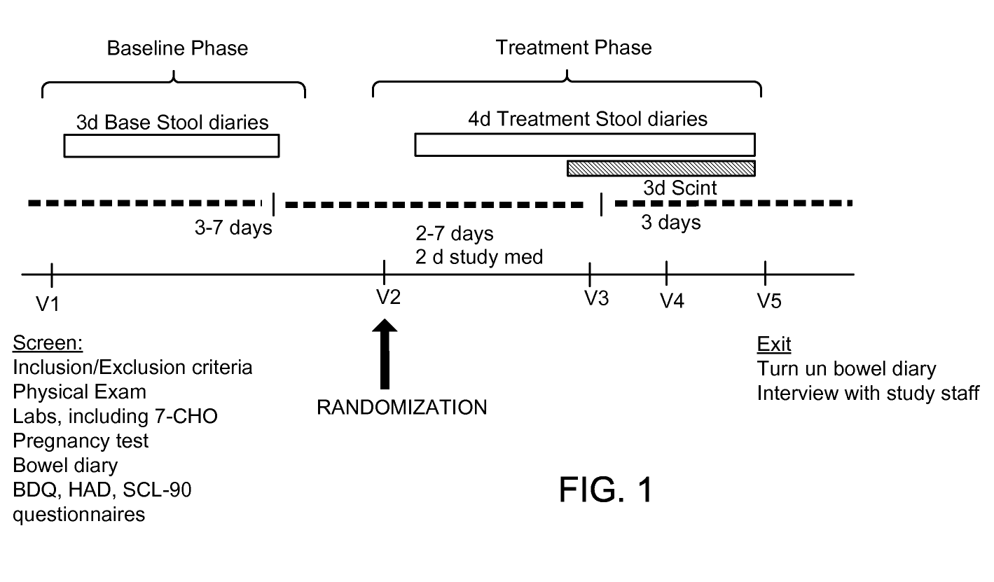

Gastrointestinal (GI) and colonic transit was conducted by a scintigraphic method (Burton et al., J. Nucl. Med., 38:1807-10 (1997); Cremonini et al., Aliment Pharmacol. Ther., 16:1781-90 (2002); and Camilleri and Zinsmeister, Gastroenterology, 103:36-42 (1992)) during the last 48 hours of drug ingestion (FIG. 1).

### Gastrointestinal Transit Measurements

An adaptation of an established scintigraphic method was used to measure GI and colonic transit (Burton et al., J. Nucl. Med., 38:1807-10 (1997); Cremonini et al., Aliment Pharmacol. Ther., 16:1781-90 (2002); and Camilleri and Zinsmeister, Gastroenterology, 103:36-42 (1992)). ¹¹¹In was adsorbed on to activated charcoal particles and delivered to the colon by means of a methacrylate-coated, delayed-release oral capsule. The capsule was ingested following an overnight fast. After the capsule emptied from the stomach, a ^{99m}Tc-sulfur colloid radiolabeled meal was ingested. It consisted of two scrambled eggs, one slice of whole wheat bread, and one glass of whole milk. This meal facilitates measurement of gastric and small bowel transit. Subjects ingested standardized meals for lunch and dinner at 4 and 8 hours after the radiolabeled meal, respectively. Abdominal scans were obtained every hour for the first 6 hours (the first 4 hours for the assessment of gastric emptying) and at 8, 24, and 48 hours after ingestion of the ¹¹¹In capsule. The performance characteristics of this test are summarized elsewhere (Cremonini et al., Aliment Pharmacol. Ther., 16:1781-90 (2002)).

### Transit Data Analysis

The counts in the stomach and each of four colonic regions: ascending, transverse, descending, and combined sigmoid and rectum were quantitated with a variable region of interest program. Counts were corrected for isotope decay, tissue attenuation, and downscatter of ¹¹¹In counts in the ^{99m}Tc window (Burton et al., J. Nucl. Med., 38:1807-10 (1997) and Cremonini et al., Aliment Pharmacol. Ther., 16:1781-90 (2002)).

Gastric emptying t_{1/2} is a measure of the time for 50% of the radiolabeled meal (identifiable by radiolabeled tracer) to empty from the stomach. Colonic filling at 6 hours, or the proportion of the radiolabeled meal to have reached the colon at 6 hours is an indirect measurement of small bowel transit time. Overall colonic transit was summarized as the colonic geometric center (GC) at specified times. The GC is the weighted average of counts in the different colonic regions [ascending (AC), transverse (TC), descending (DC), rectosigmoid (RS)] and stool, respectively 1 to 5. Thus, at any time, the proportion of counts in each colonic region is multiplied by its weighting factor as follows:

(% AC×1+% TC×2+% DC×3+% RS×4+% stool×5)/100=GC

Thus, a higher GC reflects a faster colonic transit. Ascending colon emptying was summarized by the t_{1/2} calculated by linear interpolation of values on the AC emptying curve.

The primary endpoints were the colonic GC at 24 hours (GC 24) and AC emptying t_{1/2}. Secondary transit endpoints were GC at 48 hours, the gastric emptying t_{1/2}, and the colonic filling at 6 hours. Colonic GC is an important endpoint which has been shown to be responsive to treatment with prokinetics such as prucalopride (Bouras et al., Gastroenterology, 120:354-60 (2001)), tegaserod (Prather et al., Gastroenterology, 118:463-8 (2000)), and renzapride (Camilleri et al., Clin. Gastroenterol. Hepatol., 2:895-904 (2004)) in previous pharmacodynamic studies using the same methods in patients with constipation predominant IBS or functional constipation.

### Daily Stool Diaries

During at least 3 days of the baseline period and the 4 days of treatment period, each patient noted each bowel movement with the exact time and with the description of stool consistency according to the BSFS (ranging from 1=“hard lumps” to 7=“watery”), and the ease of passage (ranging from 1=“manual disimpaction” to 7=“incontinence”), and answered whether or not they felt they had completely emptied their bowels (1=“yes” and 0=“no”) (Bouras et al., Gastroenterology, 120:354-60 (2001)). The diaries contained values for stool consistency, stool frequency, ease of passage and sense of completely emptying their bowels.

### 7α-HCO(C4) Measurements

The measurement of serum 7α-hydroxy-4-cholesten-3-one (7α-HCO or C4), which is a measurement of hepatic cholesterol synthesis and is closely related to the fecal loss of bile acids, is a validated method for BAM (Sauter et al., Dig. Dis. Sci., 44:14-9 (1999); and Gälman et al., J. Lipid Res., 44:859-66 (2003)). Participants had fasting plasma 7α-HCO measured to assess for underlying bile acid malabsorption.

### Statistical Analysis

The primary endpoints for analysis were colonic GC 24 h and ascending colon emptying T_{1/2}. An analysis of covariance (ANCOVA) assessed the treatment effects of chenodeoxycholate dose on the endpoints listed above, with age, gender and BMI, as covariates. The ANCOVA analysis compared the responses overall among the three (randomly assigned) treatment groups. Specific pairwise comparisons (e.g. each dose of chenodeoxycholate against placebo) were also examined.

### Sample Size Assessment

Sample size assessment (Table 1) was based on the results of primary endpoints in healthy volunteers (data show mean±SD). The estimated effect sizes are based on a 2-sample t-test with N=20 per group, where effect size is the difference in group means as a percentage of the corresponding overall mean (shown in Table 1). Note that the effect size demonstrable for colonic GC24 hours and for ascending colon T_{1/2} was 34% and 50% respectively. Moreover, the observed variation (CV %) in these two primary endpoints in the subjects randomized to placebo was 39% and 54%, very close to the a priori assumed values (Table 1). It was anticipated that the ANCOVA analyses would provide similar power for somewhat smaller effect sizes by pooling residual variation across all three treatment groups and by incorporating relevant covariates.

TABLE 1
Pooled Data Used to Determine Effect Size Demonstrable
with 20 participants per treatment group

| | | | | Effect size (%) |
|---|---|---|---|---|
| | | | COV | demonstrable with 80% power, |
| | Mean | SD | (%) | α = 0.05, n = 20 per group |
| Ascending colon | 15.4 | 8.5 | 55 | 50 |
| t½, h | | | | |
| Colon GC 24 h | 2.05 | 0.77 | 38 | 34 |

### Results

### Participants, Study Conduct and Completion

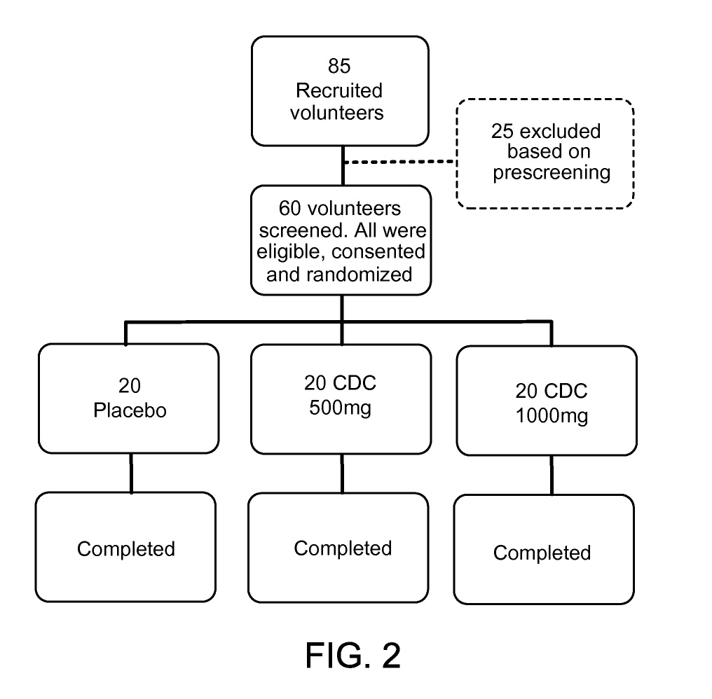

Eighty-five volunteers were recruited for the study through advertisements and mail notifications (FIG. 2). Medical records were screened for major exclusion criteria (i.e., prior GI surgery and concomitant medications). Twenty-five were ineligible based on this initial screen. Of those eligible to participate, 60 fulfilled the inclusion/exclusion criteria, consented, and were randomized. Demographic data of all randomized patients are shown in Table 2. The chenodeoxycholate and placebo groups were similar regarding age and BMI. 60 randomized patients completed the study. Patients took all of the study medications.

TABLE 2
Demographics of Participants in Three
Treatment Groups (mean ± SEM)

| | Placebo | CDC 500 mg | CDC 1000 mg |
|---|---|---|---|
| N | 20 (14 female) | 20 (15 female) | 20 (14 female) |
| Age, y | 34.6 ± 2.2 | 41.6 ± 1.9 | 40.1 ± 2.7 |
| BMI, Kg/m² | 26.3 ± 1.0 | 26.1 ± 1.0 | 26.4 ± 1.0 |

### Bile Acid Malabsorption and 7αHCO(C4) Measurements

In the laboratory, a value of <61 ng/mL is established and validated as a normal 7αHCO level to exclude BAM (Camilleri et al., Neurogastroenterol. Motil., 21(7):734-43 (2009)). Fifty-five (92%) out of the 60 participants had normal values.

### Effect of Chenodeoxycholate on Gastrointestinal and Colonic Transit

### Gastric and Small Bowel Transit

Treatment effects of CDC on gastric emptying and colonic filling were not detected (Table 3).

TABLE 3
Effects of CDC on GI Transit and Bowel Functions (mean ± SEM)

| | Placebo | CDC 500 mg | CDC 1000 mg |
|---|---|---|---|
| | N = 20 | N = 20 | N = 20 |
| GE t_{1/2} (min) | 122.8 ± 6.1 | 126.9 ± 5.3 | 143.0 ± 14.1 |
| CF 6 (%) | 54.6 ± 6.8 | 49.6 ± 7.0 | 46.0 ± 7.1 |
| GC 4 | 0.97 ± 0.24 | 0.87 ± 0.18 | 1.27 ± 0.33 |
| GC 6 | 1.28 ± 0.26 | 1.53 ± 0.28 | 2.20 ± 0.43 |
| GC 8 | 1.43 ± 0.25 | 1.83 ± 0.27 | 2.52 ± 0.39 |
| GC 24* | 2.69 ± 0.24 | 2.80 ± 0.27 | 3.76 ± 0.30 |
| GC 48** | 3.76 ± 0.20 | 4.10 ± 0.21 | 4.92 ± 0.05 |
| AC t_{1/2} (h) | 14.5 ± 1.7 | 12.1 ± 2.1 | 10.7 ± 1.9 |
| Stool Frequency | 1.09 ± 0.13 | 1.50 ± 0.18 | 2.01 ± 0.15 |
| per day # | | | |
| Stool consistency by | 3.51 ± 0.16 | 4.29 ± 0.19 | 4.80 ± 0.15 |
| Bristol Stool Form | | | |
| Scale** | | | |
| Ease of passage | 3.9 ± 0.03 | 4.1 ± 0.06 | 4.3 ± 0.06 |
| (scale 1-7)** | | | |

*p = 0.01;
**p < 0.0001;
# p < 0.001

### Overall Colonic Transit Time Assessed by Geometric Center

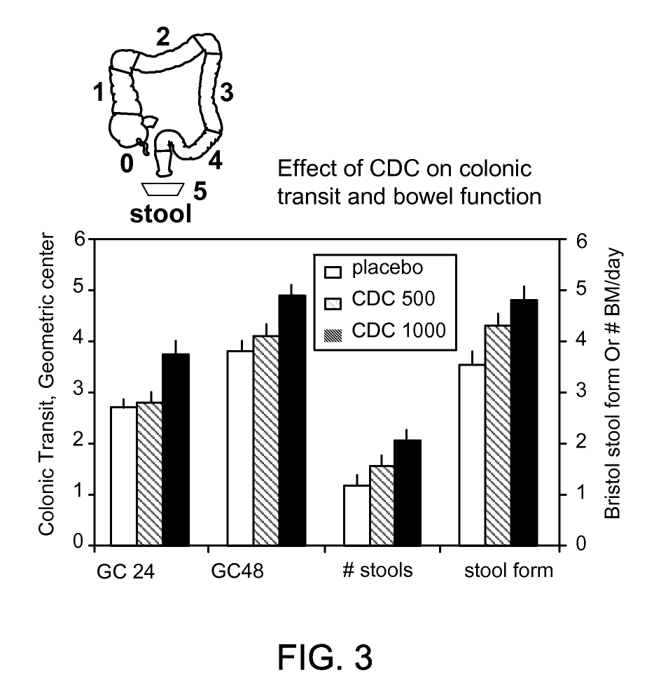

Treatment effects on overall colonic transit were significant at 24 and 48 hours (ANCOVA P=0.01 and <0.0001 respectively), as detailed in Table 3 and illustrated in FIG. 3. The effect of the 1000 mg dose was significantly greater than the 500 mg dose on the study primary endpoint, colonic GC at 24 hours.

### Effect of Chenodeoxycholate on Bowel Function

There were also significant overall treatment effects of chenodeoxycholate on stool frequency, consistency, ease of passage (all p<0.001) with a sense of incomplete evacuation (p=0.02).

### Adverse Events

The most common adverse events were diarrhea and lower abdominal cramps, which occurred more frequently in the chenodeoxycholate 1000 mg group. There were no serious adverse events (AE), and no patient had to stop treatment due to an adverse event (Table 4).

TABLE 4
Percent of Participants with Each Adverse Effect Recorded in the Entire Study Population

| | Loose | | Lower Abdo | | | Lower abdo | | Muscle | | Dizzy, light- | Sensation | |
|---|---|---|---|---|---|---|---|---|---|---|---|---|
| Drug | stools | Diarrhea* | pain/cramps | Nausea | Gas | bloating | Headache | aches | URI | headed | of warmth | Sweating |
| Placebo | 0 | 0 | 0 | 0 | 5 | 5 | 20 | 5 | 5 | 0 | 0 | 0 |
| CDC 500 mg | 15 | 40 | 15 | 5 | 5 | 5 | 25 | 5 | 0 | 10 | 5 | 5 |
| CDC 1000 mg | 20 | 75 | 75 | 20 | 5 | 0 | 25 | 0 | 10 | 5 | 5 | 5 |

*p < 0.05 by Chi-square vs. placebo; Feeling of fullness, nasal congestion, anxiety, sore throat, abnormal low body temperature, feeling of weakness and rectal pain were each experienced by one participant.

This study examined the effect of chenodeoxycholate on gastrointestinal and colonic transit in healthy volunteers. Sodium chenodeoxycholate, a di-α hydroxy bile salt, given at doses used for gallstone dissolution, accelerated whole colonic transit. Both doses of chenodeoxycholate (500 mg and 1000 mg) tested exhibited a significant treatment effect on overall colonic transit, but the higher dose was more effective. There was not a significant effect of chenodeoxycholate on ascending colon emptying rate, despite the numerical trend for a shorter half-emptying time with increased doses of the bile salt relative to placebo. The lack of a significant effect on ascending colon emptying may represent a type II statistical error; note in Table 1 that the effect size with the selected number of participants in each group was 50% for ascending colon emptying and 34% for colon GC at 24 hours.

The acceleration in colonic transit was accompanied by a looser stool form and increased stool frequency and ease of passage. Only a small minority of participants had evidence suggestive of bile acid malabsorption at baseline. Ileocolonic delivery of chenodeoxycholate did not have a significant effect on gastric emptying or colonic filling at 6 hours, a measure of orocecal transit. Given the lack of effect on gastric emptying and orocecal transit, it can be inferred that chenodeoxycholate did not alter small bowel transit.

In this study, a methacrylate-coated capsule was used to deliver chenodeoxycholate to the ileocolonic region, and doses of the bile acid that were in the lower half of the dose range previously used in the dissolution of gall stones (about 7 to 14 mg kg⁻¹day⁻¹) were used.

The optimal dose of chenodeoxycholate for the treatment of constipation is unclear from efficacy and safety perspectives. From an efficacy perspective, there were no prior studies of the ileocolonic delivery on colonic transit in humans. The results provided herein provide information on the doses that can be used in patients.

### Example 2

### Dose-Related Effects of Chenodeoxycholate on Gastrointestinal and Colonic Transit and Bowel Function in Female Patients with Constipation-Predominant Irritable Bowel Syndrome

The following study was performed to evaluate the effects of ileocolonic delivery of sodium chenodeoxycholate (CDC) on gastrointestinal (GI) and colonic transit and bowel function in constipation-predominant irritable bowel syndrome (IBS-C). A double-blind, placebo-controlled study evaluated effects of once-daily CDC for 4 days in 36 female IBS-C patients (mean age 41.80±1.62 y). Patients were randomized to oral placebo, 500 mg or 1000 mg CDC delivered to the ileocolonic region in pH-sensitive, methacrylate-coated capsules. Fasting serum 7α-hydroxy-4-cholesten-3-one (7αC4) was measured to assess for baseline abnormalities in bile acid synthesis. GI and colonic transit (primary endpoints: colonic geometric center (GC) at 24 hours (GC 24) and ascending colon (AC) t_{1/2} (AC t_{1/2})) were evaluated by scintigraphy during the last 48 hours of drug ingestion, and bowel function by validated daily questionnaires including Bristol Stool Form Scale. Treatment effects were compared by ANCOVA with BMI as a covariate, and each CDC dose against placebo by Dunnett's test.

Baseline serum 7αC4 was normal (<61 ng/mL) in 35 of the 36 patients. Gastric emptying t_{1/2} and colonic filling at 6 hours were not affected (Table 5). There was a significant overall CDC versus placebo contrast for colonic GC 24 and AC t_{1/2}, ANCOVA, p=0.005 and p=0.028, respectively; a dose-related response was observed (Table 5). Thus, the GC24 is greater with 1000 mg dose than 500 mg dose of CDC treatment showing that isotope had traveled through a larger portion of the colon at 24 hours with the higher dose and both were significantly different from placebo. Similarly, the time taken for half the isotope to empty from the ascending colon was shorter with 1000 mg CDC than with 500 mg CDC, and both were faster than with placebo treatment. For GC 24, CDC 1000 mg differed from placebo (p=0.012), with CDC 500 mg being borderline (p=0.066). For AC t_{1/2}, only CDC 1000 mg differed from placebo (p=0.058). A significant overall treatment effect was also found for stool consistency (p=0.032), but not stool frequency, ease of passage, or sense of complete evacuation. The most common side effect was lower abdominal cramps/pain (45% of CDC 500 mg and 42% of CDC 1000 mg patients). Diarrhea occurred in 18% and 17% patients on CDC 500 mg and 1000 mg, respectively. No safety issues were identified.

These results demonstrate that ileocolonic delivery of CDC accelerates colonic transit (including AC) and loosens stool form. Ileocolonic delivery of CDC can be used in the treatment of bowel dysfunction in IBS-C.

TABLE 5
Effects of Treatments on Transit and Bowel Function (mean ± SEM);
P value refers to CDC vs. placebo contrasts.

| | Placebo | CDC 500 mg | CDC 1000 mg | |
|---|---|---|---|---|
| | N = 13 | N = 11 | N = 12 | P |
| CF 6 (%) | 50.62 ± 6.73 | 52.64 ± 6.67 | 54.75 ± 7.05 | ns |
| GC 24 | 2.19 ± 0.19 | 3.12 ± 0.43 | 3.49 ± 0.36 | 0.005 |
| AC t_{1/2} (h) | 15.77 ± 2.45 | 9.55 ± 2.90 | 8.19 ± 1.78 | 0.028 |
| Stool frequency | 1.06 ± 0.16 | 1.22 ± 0.19 | 1.27 ± 0.13 | ns |
| per day | | | | |
| Stool | 2.83 ± 0.23 | 3.58 ± 0.33 | 3.47 ± 0.23 | 0.032 |
| consistency | | | | |
| (Bristol Scale) | | | | |

### Example 3

### Capsule Measurements

The thicknesses of gelatin capsules before and after being coated with a single 5 second dip into a solution having 13 g of methacrylate in 100 mL of a 4:6 ratio of acetone to isopropyl alcohol were measured. The enface view of the blank gelatin capsules demonstrated an average thickness of about 130 μm. After applying the methacrylate coating to three capsules using the single dip process, each bisected capsule was visualized enface and measured at four points. The thickness of the methacrylate coating ranged from 12 μm to 75 μm with the average being about 50 μm.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a constipation condition, said method comprising administering to a mammal having said constipation condition a composition comprising a bile acid compound, wherein said composition is configured for the delayed-release of said bile acid compound to the ileocolonic region of said mammal.

2. The method of claim 1, wherein said mammal is a human.

3. The method of claim 1, wherein said constipation condition is occasional constipation, chronic constipation, functional constipation, opiate-induced constipation, chronic colonic pseudoobstruction, slow transit constipation, or colonic inertia.

4. The method of claim 1, wherein said bile acid compound is sodium chenodeoxycholate.

5. The method of claim 1, wherein said composition comprises sodium chenodeoxycholate coated with methacrylate.

6. A composition comprising sodium chenodeoxycholate coated with methacrylate such that said sodium chenodeoxycholate is capable of being delivered to the ileocolonic region of a mammal following an oral administration.

7. The composition of claim 6, wherein said composition comprises between 250 and 5000 mg of said sodium chenodeoxycholate.

8. The composition of claim 6, wherein said composition comprises between 500 and 1500 mg of said sodium chenodeoxycholate.

9. The composition of claim 6, wherein said composition comprises about 1000 mg of said sodium chenodeoxycholate.

10. The composition of claim 6, wherein said coating is between 10 μm and 90 μm in thickness.

11. The composition of claim 6, wherein said coating is between 12 μm and 75 μm in thickness.

12. The composition of claim 6, wherein said coating is between 40 μm and 60 μm in thickness.

13. The composition of claim 6, wherein said coating is about 50 μm in thickness.

14. The composition of claim 6, wherein said sodium chenodeoxycholate is within a gelatin capsule, and said gelatin capsule is coated with said methacrylate.

15. The composition of claim 14, wherein said gelatin capsule has a thickness of between about 100 μm and about 160 μm.

16. The composition of claim 14, wherein said gelatin capsule has a thickness of between about 110 μm and about 150 μm.

17. The composition of claim 14, wherein said gelatin capsule has a thickness of between about 120 μm and about 140 μm.
